(19) United States
(12) Patent Application Publication
(10) Pub. No.: US 2006/0040311 A1
(43) Pub. Date: Feb. 23, 2006

(54) Integrated cartridge for sample manipulation

(75) Inventors: Bradley Thomas, Timonium, MD (US); Alan E. Harper, Jamul, CA (US); Charles E. Clemens, Encinitas, CA (US); Raymond D. Clark, Oceanside, CA (US); Robert J. Elms, San Diego, CA (US); Lanny Gorton, San Diego, CA (US); John B. Slate, San Diego, CA (US); Richard P. Meyst, Valley Center, CA (US)

Correspondence Address: DAVID W HIGHET VP AND CHIEF IP COUNSEL;BECTON DICKINSON AND COMPANY, 1 BECTON DRIVE, MC110, FRANKLIN LAKES, NJ, 07417-1880, US

(73) Assignee: Becton Dickinson and Company, Franklin Lakes, NJ (US)

(21) Appl. No.: 11/258,123

(22) Filed: Oct. 26, 2005

Related U.S. Application Data
(63) Continuation of application No. 10/160,191, filed on Jun. 4, 2002, now abandoned.

(51) Int. Cl. C12Q 1/68; C12M 1/34

(52) U.S. Cl. 435/6; 435/287.2

(57) ABSTRACT

An integrated cartridge for automated sample manipulation and, particularly strand displacement amplification, is provided. The cartridge comprises a sealed, two-part device with internal fluid channels and chambers, as well as reagents. The cartridge performs the sequence of fluid transfers, reagent additions and heat transitions, such as those of the strand displacement amplification process, in a single, sealed device.

### BACKGROUND OF THE INVENTION

1. Field of the Invention

The present invention relates to a device and method for the chemical processing of a biological sample and, more particularly, to an integrated cartridge for sample manipulation. The integrated cartridge comprises a sealed, two-part device with various internal fluid channels and chambers, some of which contain dried reagents. The integrated cartridge is capable of manipulating small liquid volumes through movement, reagents, filtering and heat. The integrated cartridge is well suited for process samples for high throughput screening and is particularly useful for performing assays such as nucleic acid sequence amplification.

2. Discussion of the Background

Determining the nucleic acid sequence of genes is important in many situations. For example, numerous diseases are caused by or associated with a mutation in a gene sequence relative to the normal gene. Such mutation may involve the substitution of only one base for another, called a “point mutation.” In some instances, point mutations can cause severe clinical manifestations of disease by encoding a change in the amino acid sequence of the protein for which the gene codes. For example, sickle cell anemia results from such a point mutation.

Other diseases are associated with increases or decreases in copy numbers of genes. Although the determination of the presence or absence of changes in a sequence is important, determination of the quantity of such sequences in a sample can be used in the diagnosis of disease or the determination of the risk of developing disease. Moreover, variations in gene sequences of both prokaryotic and eukaryotic organisms has proven invaluable to identifying sources of genetic material (e.g., identifying one human from another or the source of DNA by restriction fragment length polymorphism).

Certain infections caused by microorganisms or viruses may also be diagnosed by the detection of nucleic acid sequences peculiar to the infectious organism. Detection of nucleic acid sequences derived from viruses, parasites and other microorganisms is also important where the safety of various products is of concern, e.g., donated blood, blood products and organs in the medical field and the safety of food and water supplies.

Identification of specific nucleic acid sequences by the isolation of nucleic acids from a sample and detection of the sought for sequences provides a mechanism whereby one can determine the presence of a disease, organism or individual. Generally, such detection is accomplished by using a synthesized nucleic acid “probe” sequence that is complimentary in part to the target nucleic acid sequence of interest.

Although it is desirable to detect the presence of nucleic acids as described above, it is often the case that the sought for nucleic acid sequences are present in sample sources in extremely small numbers. The condition of small target molecule numbers causes a requirement that laboratory techniques be performed in order to amplify the numbers of the target sequences so that they may be detected. There are many known methods of amplifying targeted sequences. One such method is strand displacement amplification (SDA).

The current format for performing SDA procedures is problematic for several reasons. First, SDA is currently performed in microtiter well plates. Such procedure typically uses two 96-well plate to process 94 liquid patient samples. The first plate, which is where the prime reaction takes place, typically contains short DNA sequences known as “primers” needed to initiate the subsequent amplification reaction, which occurs in the wells of the second(i plate. The two plates must be placed on separate heat blocks because the prime and amplification (“amp”) reactions require different temperatures. Furthermore, the user must work quickly after transferring the sample to the amplification plate because there is a time limit for placing the plate in the reader once amplification starts.

Another problem with existing methods for performing SDA procedures is the requirement of manual pipetting. Currently, the user must transfer the liquid samples from the sample tube to the prime plate and later to the amplification plate using a multi-channel, manual pipette. Manual pipetting is tedious and time consuming because each transfer must be held steady for a few seconds while the pipette mixes the liquid in the wells. Manual pipetting also requires the user to keep track of which row of the plate he or she is on.

A need, therefore, exists for an automated sample manipulation cartridge and, more particularly, for an automated SDA DNA amplification process in a miniaturized and sealed, disposable sample manipulation cartridge so as to overcome the deficiencies of current, manual SDA methods.

### SUMMARY OF THE INVENTION

The present invention relates to a device for automated sample manipulation. In particular, the present invention relates to a miniaturized and sealed disposable device for the automation of sample manipulation, especially the SDA DNA amplification process. The device of the present invention performs a sequence of fluid transfers, reagent additions and heat transitions in a unitized package. The device comprises a disposable cartridge and a means for driving the fluidics and heating functions of the cartridge. The cartridge comprises a sealed, two-part device with various internal fluid channels and chambers and dried reagents. The fluid channels connect a series of chambers that are used to measure an aliquot, provide heat and reagents for reactions, and control the position of the fluid bolus for each reaction step.

The integrated cartridge of the present invention has several advantages. While the integrated cartridge of the present invention contains the same fluidic metering, fluid transfer, reagent addition and heating functionality as current manual SDA methods, the present invention, unlike current SDA methods, is automated. A major advantage of the present invention, therefore, is the ease of use. For example, sample introduction can be done with a dropper instead of a pipette because the cartridge itself meters the required sample volume. Additionally, after sample input, the user simply seals the inlet well with a sealing means and places the cartridge in a cartridge processing device; no additional liquid handling needs to be done by the user. Moreover, the sealed, integrated cartridge of the present invention is better for evaporation, aerosol containment and sealed disposal of the completed test.

The above and other features and advantages of the present invention will become more apparent from the following detailed description of the presently preferred embodiments, particularly when considered in conjunction with the drawings, and to the appended claims.

### BRIEF DESCRIPTION OF THE DRAWINGS

FIG. 1 is an unassembled view of the integrated cartridge of the present invention from the bottom thereof.

FIG. 2 is an unassembled view of the integrated cartridge of the present invention from the top thereof.

FIG. 3 is a perspective view of the integrated cartridge from the top thereof.

FIG. 4 is fragmentary sectional view of the integrated cartridge of the present invention along line A-A of FIG. 3.

FIG. 5 is an enlarged view along circle B of FIG. 4.

FIG. 6 is a fragmentary sectional view along line CC of FIG. 3.

FIG. 7 is a fragmentary sectional view of the integrated cartridge of the present invention along line D-D of FIG. 3.

FIG. 8 is an enlarged view along circle E of FIG. 7.

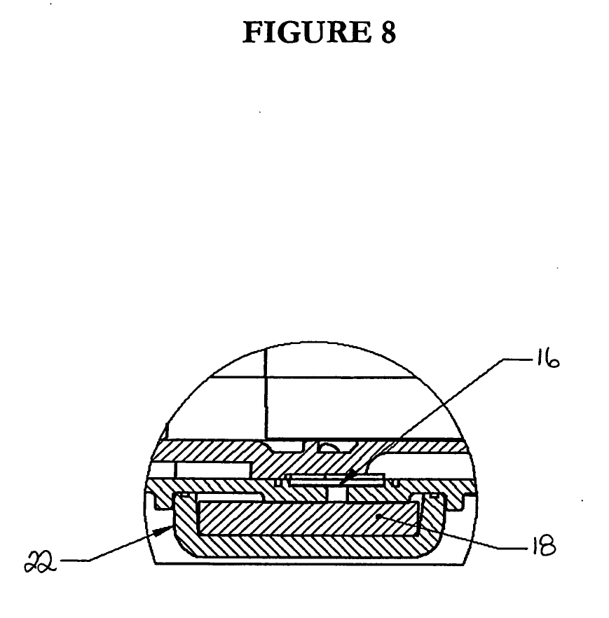

FIG. 9 shows the integrated cartridge of the present invention in a closed cartridge processing device.

FIG. 10 shows the integrated cartridge of the present invention in an open cartridge processing device.

FIG. 11 is an enlargement of the cartridge processing device showing the active components of thereof.

FIG. 12 is a schematic drawing of the cartridge of the present invention and its drivers.

### DETAILED DESCRIPTION OF THE INVENTION

While this invention is satisfied by embodiments in many different forms, there will herein be described in detail preferred embodiments of the invention, with the understanding that the present disclosure is to be considered as exemplary of the principles of the invention and is not intended to limit the invention to the embodiments illustrated and described. Numerous variations may be made by persons skilled in the art without departure from the spirit of the invention. The scope of the invention will be measured by the appended claims and their equivalents.

In describing the present invention, various terms and phrases will be used herein. Generally, the meaning of these terms are known to those having skill in the art and are further described below. The definitions should not be understood to limit the scope of the invention. Rather, they should be used to interpret language of the description and, where appropriate, the language of the claims. These terms may also be understood more fully in the context of the description of the invention. If a term is included in the description or the claims that is not defined below, or that cannot be interpreted based on its context, then it should be construed to have the same meaning as it is understood by those of skill in the art.

The term “amplification” refers to the increase in the number of copies of a particular nucleic acid target of interest.

The term “amplicon” refers to the product of an amplification reaction, i.e., the copy of a particular nucleic acid target of interest.

The term “amplification components” refers to the reaction materials such as enzymes, buffers and nucleic acids necessary to perform an amplification reaction to form amplicons of a target nucleic acid of interest.

The term “sample” refers to a substance that is being assayed for the presence of one or more nucleic acids of interest. The nucleic acid, or acids, of interest may be present in a mixture of other nucleic acids. A sample containing the-nucleic acids of interest may be obtained in numerous ways. It is envisioned that the following could represent samples: cell lysates, purified genomic DNA, body fluids such as from a human or animal, clinical samples, food samples, etc.

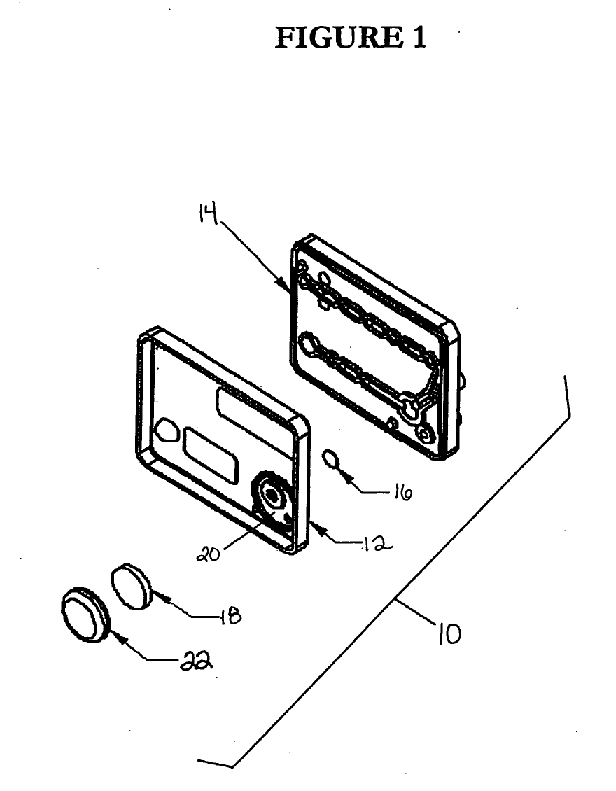

The present invention comprises a sealed, integrated cartridge with fluidic metering, fluid transfer, reagent addition and heating functionalities for sample manipulation. The cartridge of the present invention will first be described by reference to the figures. As seen in FIG. 1, the cartridge 10 of the present invention is a two-part assembly having internal fluidic cavities. Cartridge assembly 10 generally comprises a cartridge bottom 12 and a cartridge top 14. Cartridge bottom 12 generally comprises a flat plate, while cartridge top 14 generally comprises a molded plate. The plates are preferably transparent. The plates may be made of any suitable material, but preferably are acrylic and, more preferably, are made of polymethylmethacrylate (PMMA) resin.

Cartridge top 14 has fluidic features, which will be described in more detail later, including capillary channels, reaction chambers and the like, molded therein. Each reaction chamber is preferably shaped like a half cylinder with full-round ends and has a radius of about 0.060 inches. Each capillary channel, which connect the various fluid chambers, is preferably shaped like a half cylinder and has a radius of about 0.015 inches.

Cartridge 10 also comprises a filter membrane 16 located in a cavity (not shown) inside of cartridge bottom 12 and a waste trap absorbent pad 18 located in a recess 20 in the bottom of cartridge bottom 12. Absorbent pad 18 is covered by a vacuum chamber cover 22. PIG. 8 is an enlarged view showing filter membrane 16, absorbent pad 18 and vacuum chamber cover 22.

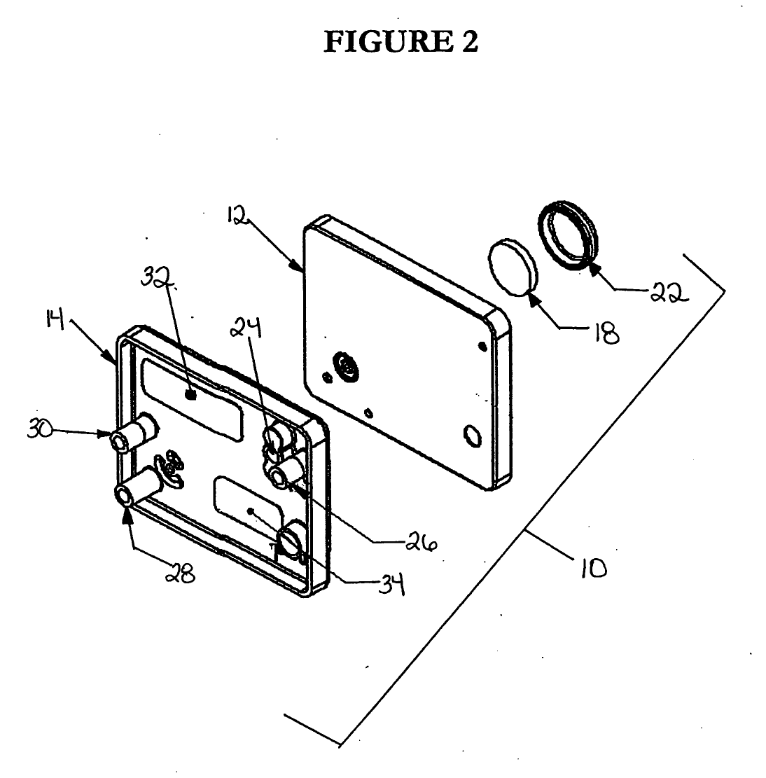

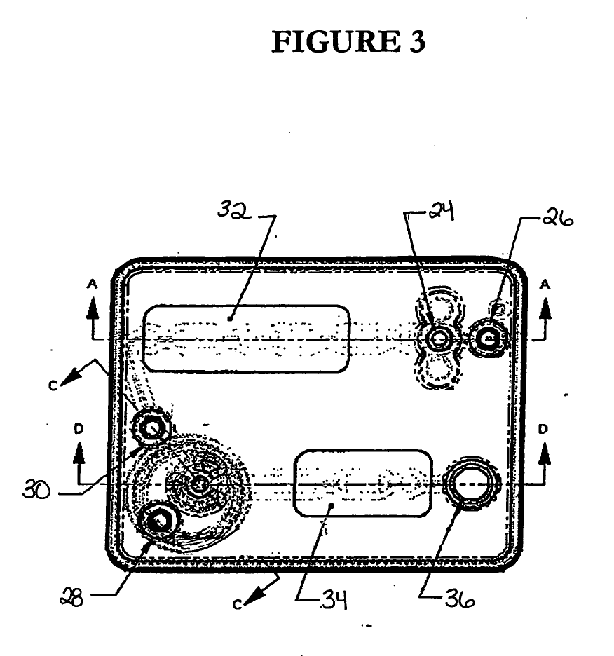

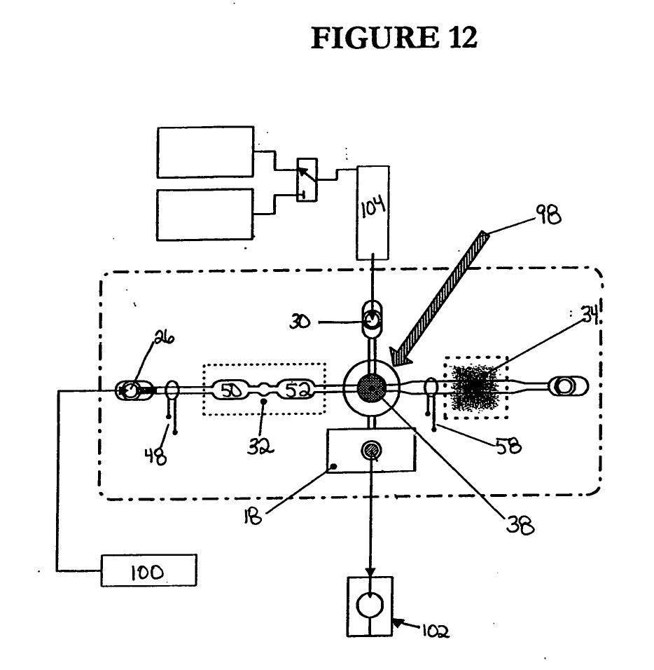

FIG. 2 is a unassembled view of cartridge 10 from the top of the cartridge. FIG. 3 is a perspective view of assembled cartridge 10 as seen from the top thereof. Cartridge top 14 includes sample inlet well 24 into which the user inputs a liquid sample. While the shape of liquid inlet well 24 is preferably cylindrical, the shape is not critical and other shapes are possible. Cartridge top 14 also includes three tapered luer ports that are used to connect fittings (not shown) for tubing from various pumping means of a cartridge processing device, which will be described in more detail later, to cartridge 10. Air drive entry port 26 is the cartridge interface point for a means of pumping air, such as an air pump 100 (FIG. 12) into the cartridge to move the liquid sample throughout the various chambers and channels. A vacuum pump 102 (FIG. 12) is connected to vacuum port 28 and is used to pull the liquid sample through filter membrane 16 into absorbent pad 18. Input port 30 is used to introduce wash buffers and other amplification components into the fluidic features of cartridge 10. Cartridge top 14 further includes a prime and amplification heating surface 32, a denature heating surface 34 and a take-out well 36.

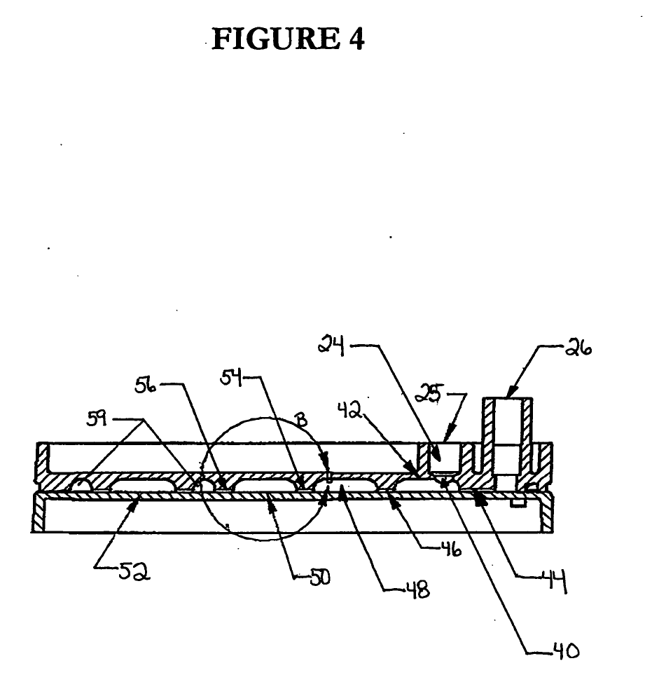

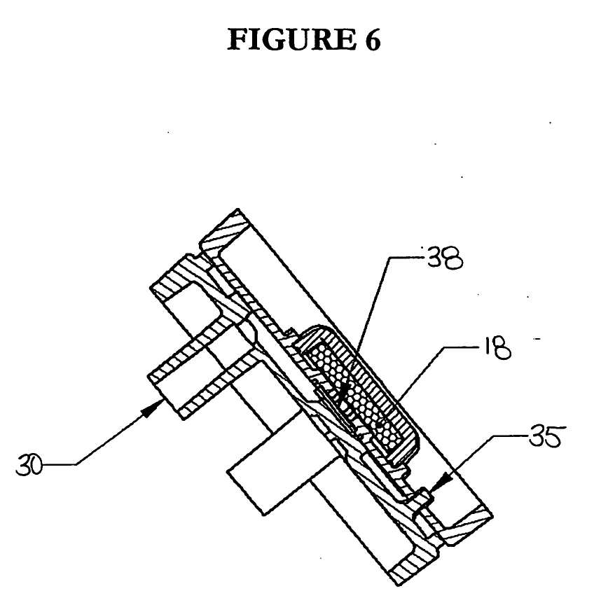

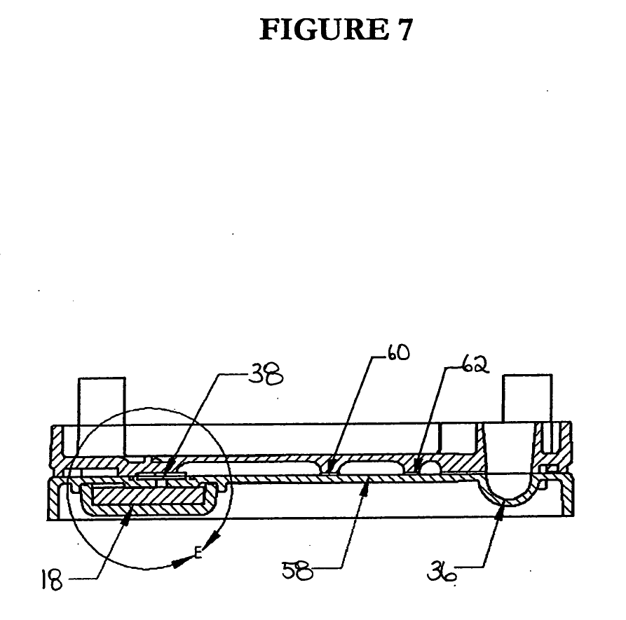

FIG. 4 is a cross-sectional, side view of the integrated cartridge of the present invention showing a portion of the fluid path. The fluid path of cartridge 10 starts with liquid inlet well 24 and flows sequentially through the various capillary channels and reaction chambers, into a desalt filter 38 (FIGS. 6 and 7) and into take-out well 36 (FIG. 3). Liquid inlet well 24 connects to a chamber entry 40. Chamber entry 40 connects to a liquid input chamber 42. On either side of liquid input chamber 42 are capillary channels 44, 46. Capillary channel 46 connects liquid input chamber 42 to a sensing chamber 48. In sensing chamber 48, the liquid sample is observed by optical through-beam sensors (not shown) to identify the meniscus and locate the leading edge of the liquid sample, or the “fluid bolus.” The curvature of the meniscus momentarily deflects the beam and causes a detectable drop in the transmission, allowing detection. Sensing chamber 48, which is of the same dimensions as the reaction chambers, contains no reagents that would disrupt the optical quality of the liquid or hinder the flow of the liquid sample.

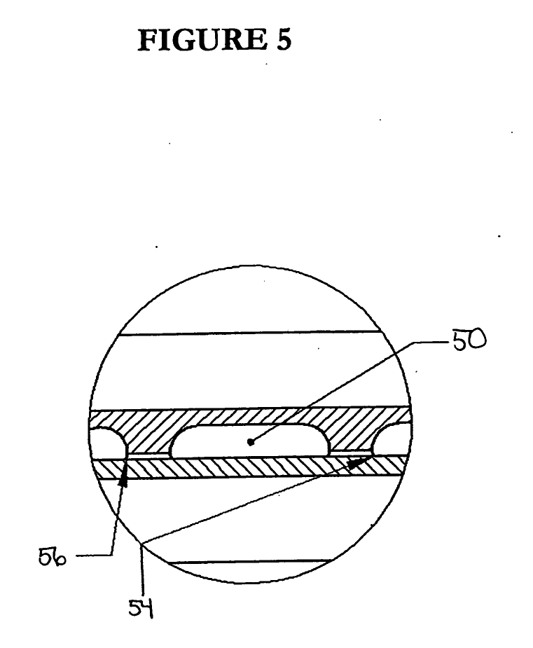

The next chambers connected in sequence by capillary channels are a prime chamber 50 and an amplification chamber 52. FIG. 5 is a detailed view of prime chamber 50 and its associated capillary channels 54, 56. Prime chamber 50 and amp chamber 52 contain reagents required for the reactions that occur therein. Preferably, the reagents are dried down in their respective chambers. The fact that the reagents are dried down in the cartridge itself can drastically change the surface wetting properties of the liquid sample, which can in turn change the flow characteristics. Typically, reagents can reduce the surface tension to the point of defeating the capillary locks. Drying down the reagents tends to eliminate this problem; however, dried coatings too close to the edges of the capillaries can still wick the fluid into the next chamber. This undesirable effect is overcome by interspersing small, uncoated, chambers 59 (FIG. 4) between the coated chambers.

FIG. 7 is a cross-sectional, side view of the cartridge assembly of the present invention showing the remaining portion of the fluid path. The remainder of the fluid path comprises desalt filter 38, which includes filter membrane 16. Located below filter membrane 16 is absorbent pad 18 to trap the liquid that comes through. The final chamber of cartridge 10 is a denature chamber 58, which preferably is used to strip apart the DNA strands into single strands with heat. Denature chamber 58 also functions as a second meniscus sensing chamber because the movement of the fluid off of filter membrane 16 is not consistent, and the leading edge of the fluid bolus must be located again. To do so, capillary channel 60 preceding denature chamber 58 is placed between the ends of a second optical sensor of the cartridge processing device to relocate the meniscus. Like sensing chamber 48 (FIG. 4), therefore, denature chamber 58 contains no reagents. Capillary channels 60, 62 are located on either side of denature chamber 58. Again, the small cross section of the capillary channels reduces evaporation, and the capillary lock feature keeps the fluid centered. After denaturing is complete and the meniscus has been relocated, the completed sample is pushed to take-out well 36 where it is manually transferred to another instrument, such as a NanoChip™ cartridge by Nanogen, Inc. of San Diego, Calif., for analysis. Instruments such as the NanoChip™ cartridge are for hybridizing and reading the amplification products but do not themselves perform the amplification process. In an alternative embodiment of the present invention, the product design would integrate the amplification cartridge with the reader so that the transfer step could be eliminated.

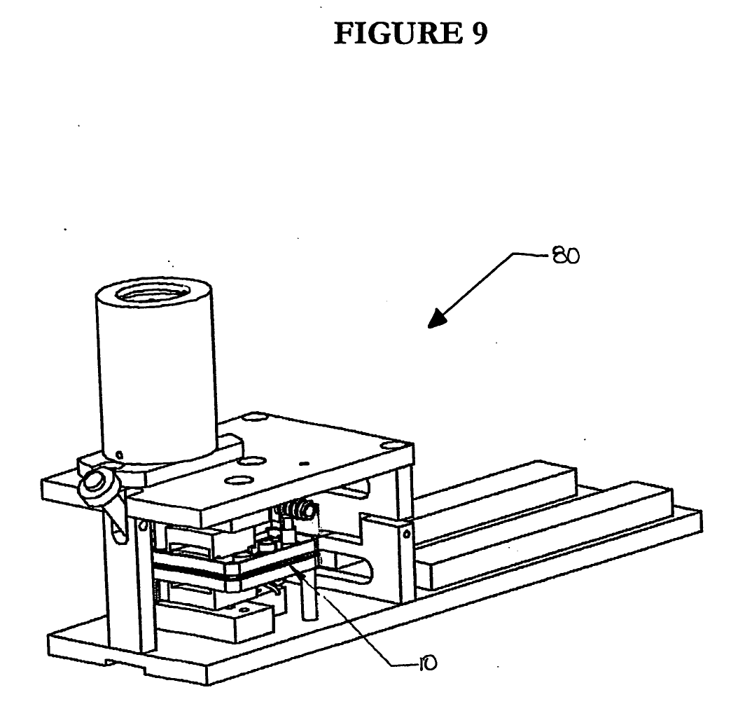

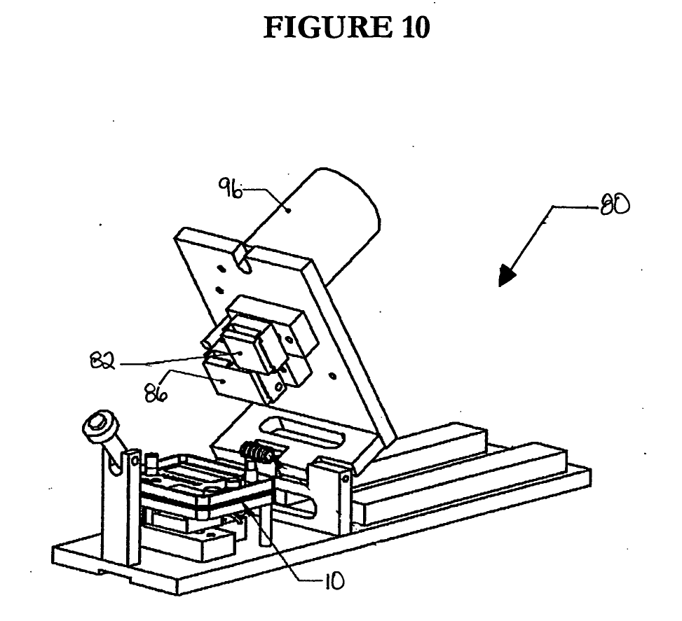

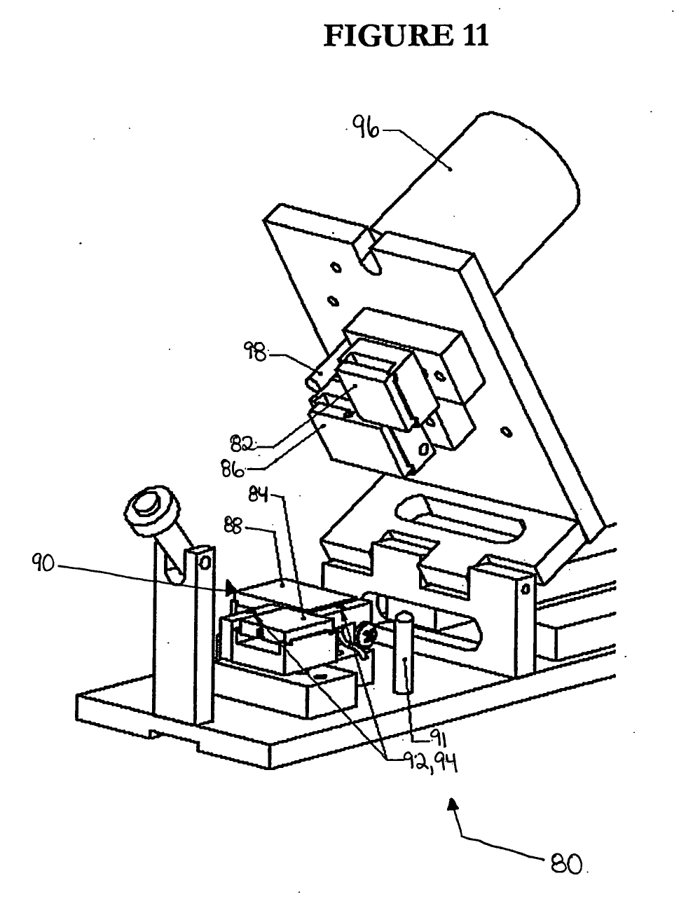

FIG. 9 shows integrated cartridge 10 of the present invention closed within a cartridge processing device 80, while FIG. 10 shows cartridge 10 in open cartridge processing device 80. FIG. 11 shows the various active components of cartridge processing device 80. Referring first to FIGS. 10 and 11, cartridge processing device 80 includes denature heat blocks 82, 84 and prime and amp heat blocks 86, 88. Cartridge 10 is placed in a cartridge nest 90 of cartridge processing device 80 such that assembly alignment pin 35 (FIG. 6) lines up with cartridge alignment pin 91 of cartridge processing device 80. Cartridge processing device 80 also includes through-beam optical sensors (“meniscus sensors”) 92, 94 that examine the non-reagent chambers to find the meniscus. These are preferably fiber optic tips. The curvature of the meniscus causes a shadow that is detectable by the sensor. Cartridge processing device 80 further includes a sonicator 96 and its associated sonicator probe 98.

Referring now to FIG. 9, when cartridge processing device 80 is closed and locked, it places the heat blocks against cartridge 10 under light spring pressure such that heat blocks 86, 88 sandwich the prime and amp chambers, while heat chambers 82, 84 sandwich the denature chamber. The heat blocks preferably are copper blocks with resistance heaters and RTD sensors that allow precise temperature control. The heat blocks are spring loaded over cartridge 10 directly over and under the reaction chambers and extending out approximately 0.250 inches on all sides of the chambers.

FIG. 12, which is a schematic drawing of the cartridge and drivers, shows the logical sequence of all the active chambers and the external driving and sensing devices.

In operation, a user places the patient sample into liquid inlet well 24. Chamber entry 40 connecting liquid inlet well 24 to liquid input chamber 42 allows the liquid sample to flow down into the chamber and fill it. When the sample reaches capillary channels 44, 46 at each end of chamber 42, it is pulled through to the opposite ends of the channels and stops. Surface tension prevents the liquid from flowing past the sharp transition from the capillary channel to the next cavity. This feature is referred to as a “capillary lock” and is described in more detail in co-pending U.S. patent application Ser. No. (Attorney Docket No. 20187-112). In general, the capillary locks allow the fluid bolus to be roughly positioned in the cavities and then self-center and lock in place.

After input chamber 42 has measured and locked the required volume for processing, the remainder of the input volume accumulates and remains in inlet well 24 above it. The user places a sealing means (not shown), preferably tape or a self-adhesive label, over the entrance 25 of inlet well 24 to form a vacuum and retain the excess liquid there when the sample in chamber 42 is moved forward. Application of a positive pressure through air drive entry port 26 moves the sample out of input chamber 42 and leaves the excess liquid trapped in inlet well 24. This self-metering input allows crude filling on the user's part, while accurate metering is performed by the self-metering volume input device. Input metering through the capillary locks and the sealing of the inlet well, therefore, eliminates the requirement for accurate pipetting. The volume of the sample processed can be varied by changing the size of the input metering chamber. This self-metering volume input device is described in more detail in copending U.S. patent application Ser. No. ______ (Attorney Docket No. 20187-113).

The reaction sequence moves the single bolus of liquid through the sequence of chambers along the capillary channels. The fluid bolus is moved into the chambers one by one where the reagents are dissolved, and the external heat blocks of the cartridge processing device maintain reaction temperatures in the reaction chambers of the cartridge.

External pumps, preferably syringe pumps, are used to move the fluid bolus through the cartridge and add reagents to it. Pump 100 connects to air drive entry port 26 adjacent liquid inlet well 24. This pump pushes only air, which moves the fluid bolus from input chamber 42 through the sensing, prime and amp chambers and onto the desalt filter. The capillary locks allow the drive fluid to be air. The compliance of air prevents accuracy in other systems and causes other systems to use deionized water as a system fluid for stiffness. The fluid movement pump moves only the volume in the liquid input chamber forward into the cartridge for processing. Any excess fluid in inlet well 24 remains trapped there by the sealing means placed over its opening 25. When the fluid movement sequence starts, pump 100 moves the fluid forward slowly into sensing chamber 48, where an optical sensor detects the meniscus. The instrument then knows the exact location of the leading edge of the liquid and proceeds with the predetermined number of steps to move the liquid into prime chamber 50. When the liquid bolus is roughly centered in prime chamber 50, the controller stops and opens a solenoid valve (not shown) to vent the tubing from the pump. This allows the capillary locks to center the bolus in prime chamber 50. The preferred method of moving the liquid is to not use the optical sensors at all. If the input chamber capillary locks function properly, then the starting position will be known, and the air volume needed to reach the prime chamber will be consistent. One or both of the meniscus sensing optics may, therefore, be eliminated by knowing the starting position of the fluid.

Prime chamber 50 contains dried-down reagents. When the fluid bolus is moved into prime chamber 50, it is held there for a specified time to allow for dissolution and reaction of the reagents. The bolus is then moved to amp chamber 52, which also contains dried-down reagents, and held there for dissolution and reaction. Both prime chamber 50 and amp chamber 52 require an elevated temperature. Heat blocks 86, 88 span the area covering these two chambers and maintain them at a constant temperature. When the fluid bolus is in the heated chambers, the capillary channels on both sides are vented to prevent pressure buildup that would move the fluid bolus out of position. The small exposed surface inside of the capillary channels also essentially eliminates evaporation.

After the amplification is complete, pump 100 moves the fluid bolus onto the face of desalt filter 38 where it passes through filter membrane 16 of desalt filter 38. Filter membrane 16 is preferably polysulfone. The capillary channels are vented, and vacuum pump 102 is started. It takes less than about 10 minutes to pull the liquid through the filter and into waste trap 18. The pore size of filter membrane 16 allows all liquids and salt ions to pass through, but traps the DNA amplicons on its surface. The amplicons tend to embed themselves into the pores because of the high fluid pressure. The amplicons are freed from the filter by agitation achieved with sonicator probe 98 pressed against the face of cartridge 10 above filter membrane 16. Input port 30 is used to add various buffers, rinse fluids and the like to the cartridge. A pump 104 uses a selector valve to draw wash buffer, preferably about 100 microliters, followed by air. By closing the valve of pump 100, the fluid introduced into input port 30 flows toward die filter and not backwards. Pump 104 pushes the wash buffer slowly onto the filter so that it can be pulled through by vacuum pump 102.

Pump 104 then uses a selector valve to push a small volume of buffer, plus the mechanical release of the sonication, to resuspend the DNA, followed by driving air onto the filter. By resuspending in a smaller volume of buffer, the DNA can also be concentrated during desalting, which increases sensitivity. If this volume is half the original sample volume, then the DNA concentration is nearly doubled (some of the DNA is lost to binding) when it is resuspended. The DNA recovery from the filter is enhanced by mechanical agitation in the form of sonication. Sonicator probe 98 is held in contact with the upper wall of the filter chamber. This is energized briefly before the resuspension buffer is moved onto the next step. The sonication improves the DNA recovery from about 50% to greater than about 80%. The wash and resuspension solutions can be varied in both composition and volume.

In the manufacture of the cartridge assembly, the prime and amp reagents are dried down in their respective reaction chambers in a vacuum oven. The filter membrane is inserted into its cavity, and a retaining ring is heat-formed down over the edge of the membrane. The two plates of the cartridge are then bonded together. Preferably, the bonding is done by silk screening an adhesive pattern onto the cartridge bottom. Silk screening is preferred because it is less abusive to the reagent dry downs than other bonding techniques, particularly ultrasonic welding. The adhesive pattern is about 0.005 inches thick and matches the outline of the walls of the molded plate. The pattern is preferably set back from the inside edges of the channels by about 0.020 inches so that it does not squeeze into them during assembly. The two plates are then clamped together and exposed to ultraviolet light to cure the adhesive. After the bonding is complete, the waste trap absorbent pad is placed into its recess in the bottom of the assembled cartridge, and the vacuum chamber cover glued on over it, preferably with the same ultraviolet adhesive. Porous, sintered plastic plugs may then be pressed into the luer ports in the cartridge top to prevent liquid contamination of the driving instrument. The assembled cartridge is packaged, preferably with a desiccant sachet, in a foil laminate pouch as a light and moisture barrier.

Having now fully described the invention with reference to certain representative embodiments and details, it will be apparent to one of ordinary skill in the art that changes and modifications can be made thereto without departing from the spirit or scope of the invention as set forth herein.

1. A device for automated sample manipulation, comprising: a liquid inlet well for providing a test sample; a liquid input chamber for receiving said test sample from said liquid inlet well; a first reaction chamber for performing a first reaction on said test sample; a second reaction chamber for performing a second reaction on said test sample; a first heating surface for heating said first and second reaction chambers; and a denature chamber for denaturing said test sample.

2. The device of claim 1, wherein the sample manipulation is DNA amplification.

3. The device of claim 2, wherein the DNA amplification is strand displacement amplification.

4. A device for automated DNA amplification, comprising: a liquid inlet well for providing a test sample containing DNA; a liquid input chamber for receiving said test sample from said liquid inlet well; a first reaction chamber for performing a prime reaction on the DNA in said test sample; a second reaction chamber for performing an amplification reaction on the DNA in said test sample; a first heating surface for heating said first and second reaction chambers; and a denature chamber for denaturing the DNA in said test sample.

5. The device of claim 4, further comprising: a first sensing chamber between the liquid input chamber and the first reaction chamber, wherein said sensing chamber is used for locating the test sample.

6. The device of claim 5, wherein the denature chamber is also used as a second sensing chamber.

7. The device of claim 4, wherein said first reaction chamber and said second reaction chamber contain reagents.

8. The device of claim 7, wherein said reagents are dried in said chambers during manufacture of said device.

9. The device of claim 4, further comprising a fluid transfer mechanism for transferring the sample from the sample input chamber to the first reaction chamber.

10. The device of claim 9, wherein said fluid transfer mechanism comprises at least one capillary channel.

11. The device of claim 4, further comprising a fluid transfer mechanism for transferring the sample from the first reaction chamber to the second reaction chamber.

12. The device of claim 4, wherein the DNA amplification is strand displacement amplification.

13. The device of claim 4, wherein said device is disposable.

14. A device for automated sample manipulation, comprising a sealed cartridge having a cartridge top and a cartridge bottom, wherein said cartridge top comprises: a liquid inlet well; fluidic cavities; first, second and third connection ports; first and second heat block surfaces; and a take-out well.

15. The device of claim 14, wherein the sample manipulation is DNA amplification.

16. The device of claim 15, wherein the DNA amplification is strand displacement amplification.

17.-27. (canceled)
